(19) United States
(12) Patent Application Publication
(10) Pub. No.: US 2006/0023831 A1
(43) Pub. Date: Feb. 2, 2006

(54) X-ray CT image processing method and X-ray CT apparatus

(75) Inventors: Yasuhiro Imai, Tokyo (JP); Akihiko Nishide, Tokyo (JP); Kazuhiko Sato, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/181,560

(22) Filed: Jul. 14, 2005

(30) Foreign Application Priority Data
Jul. 29, 2004 (JP) 2004-222065

(51) Int. Cl. H05G 1/60; A61B 6/00; G01N 23/00; G21K 1/12

(52) U.S. Cl. 378/4

(57) ABSTRACT

A method to optimize the image quality for each specified area in a tomographic image. The method includes creating a plurality of reconstruction images reconstructed by using different reconstruction functions specifying some specific areas included in the tomographic image such as organs by either the trace function, by the automatic segmentation, or by the manual correction of the result from the automatic segmentation synthesizing by switching the reconstruction images for each specified area and convoluting an image filter for each area by switching among filters. The image quality for each area or organ in a tomographic image can be thereby optimized.

### BACKGROUND OF THE INVENTION

The present invention relates to an X-ray computer tomographic imaging apparatus, more specifically to an X-ray CT image processing method and an X-ray CT apparatus for optimizing the image quality to a specific area or organ.

The conventional X-ray CT apparatus typically convolutes, for one entire tomographic image, one set of reconstruction function to the projection data, and at maximum one set of image filter or no image filter to the tomographic image (see, Patent Document 1). In this case, in one tomographic image, there exists only image quality determined by the reconstruction function and by the image filter in some cases. Although an optimum image can be provided for an organ, the image is not optimum for another organ.

[Patent Document 1] JP-A-2000-200340.

When reading the image, it is preferable to optimize the image quality and texture for each organ for ease of reading.

### SUMMARY OF THE INVENTION

Therefore, an object of the present invention is to provide an X-ray CT image processing method and an X-ray CT apparatus, which optimize the image quality for such a specific area as an organ.

In first aspect, the present invention provides an X-ray CT image processing method having the steps of: irradiating X-ray to an object for acquiring projection data of the X-ray passing through the object; preprocessing the projection data thus acquired; convoluting a reconstruction function onto thus preprocessed projection data; reverse projecting the projection data having the reconstruction function convoluted to reconstruct the image of tomography; specifying a specific area within the tomographic image; and changing the image quality of the specific area to a predetermined quality.

In the X-ray CT image processing method in accordance with the above first aspect, the optimum image quality for a given area can be achieved by determining a predetermined image quality for each area.

In second aspect, the present invention provides an X-ray CT image processing method, in which the step of specifying the specific area within the tomographic image further includes the substep of: specifying an area of specific organ.

In the X-ray CT image processing method in accordance with the above second aspect, the optimum image quality for a given organ can be achieved by determining a predetermined image quality for each organ.

In third aspect, the present invention provides an X-ray CT image processing method in accordance with first or second aspect, in which the step of specifying the specific area within the tomographic image further includes the substep of: performing the area selection by using a trace function for determining the specified area by specifying a plurality of points on the tomographic image.

In the X-ray CT image processing method in accordance with the above third aspect, the trace function for specifying an area can achieve the optimum image quality for each organ.

In fourth aspect, the present invention provides an X-ray CT image processing method in accordance with either first or second aspect, in which the step of specifying the specific area within the tomographic image further includes the substep of: performing the area selection either by means of automatic segmentation, or by manually correcting the result of automatic segmentation.

In the X-ray CT image processing method in accordance with the above fourth aspect, the automatic segmentation, or the manual area selection from within the result of automatic segmentation can achieve the optimum image quality.

In fifth aspect, the present invention provides an X-ray CT image processing method in accordance with either one of first to fourth aspects, in which the step of changing the image quality of the specific area to a predetermined quality further includes the substep of: changing the image quality to a predetermined quality by using an image filter to be convoluted onto the tomographic image.

In the X-ray CT image processing method in accordance with the above fifth aspect, the image quality can be specified by adjustment of image filter, thus allowing the optimum image quality for each organ.

In sixth aspect, the present invention provides an X-ray CT image processing method in accordance with either one of first to fifth aspects, in which the step of changing the image quality of the specific area to a predetermined quality further includes the substep of: changing the image quality to a predetermined quality by using a reconstruction function to be convoluted onto the projection data.

In the X-ray CT image processing method in accordance with the above sixth aspect, the image quality can be specified by the adjustment of reconstruction function, thus allowing the optimum image quality for each organ.

In seventh aspect, the present invention provides an X-ray CT image processing method in accordance with sixth aspect, in which the step of changing the image quality of the specific area to a predetermined image quality further includes the substeps of: convoluting a plurality of reconstruction functions each different from other to the preprocessed projection data; back projecting each of projection data having a reconstruction function convoluted to form a plurality of tomographic images; and to each specific area of tomographic image, performing a weighted addition of a plurality of tomographic images created by the back projection means to form one tomographic image.

In the X-ray CT image processing method in accordance with the above seventh aspect, the image quality can be adjusted by the weighted addition of a plurality of tomographic images, allowing the optimum image quality for each organ.

In eighth aspect, the present invention provides an X-ray CT apparatus, which includes an X-ray data acquisition means for irradiating X-ray to an object to acquire the projection data of X-ray passing through the object; a preprocessing means for preprocessing the projection data acquired by the X-ray data acquisition means; a reconstruction function convoluting means for convoluting a reconstruction function onto the preprocessed projection data; a back projection means for back projecting the projection data having the reconstruction function convoluted so as to reconstruct a tomographic image; an area selector means for selecting a specific area of tomographic image; and an image quality adjustor means for adjusting the image quality of the specific area to a predetermined quality.

In the X-ray CT apparatus in accordance with the above eighth aspect, each area is adjusted to a predetermined image quality to allow the optimum image quality for each area.

In ninth aspect, the present invention provides an X-ray CT apparatus in accordance with the above eighth aspect, in which the area selector means specifies an area of a specific organ.

In the X-ray CT apparatus in accordance with the above ninth aspect, the image quality for each organ is adjusted to a predetermined level to allow the optimum image quality for each organ.

In tenth aspect, the present invention provides an X-ray CT apparatus in accordance with either eighth or ninth aspect, in which the area selector means performs the area selection by using a trace function for determining the specified area by specifying a plurality of points on the tomographic image.

In the X-ray CT apparatus in accordance with the above tenth aspect, the optimum image quality for each organ can be achieved by specifying each area by the trace function.

In eleventh aspect, the present invention provides an X-ray CT apparatus in accordance with either eighth or ninth aspect, in which the area selector means performs the area selection either by means of automatic segmentation, or by manually correcting the result of automatic segmentation.

In the X-ray CT apparatus in accordance with the above eleventh aspect, the automatic segmentation, or the manual area selection from within the result of automatic segmentation can achieve the optimum image quality.

In twelfth aspect, the present invention provides an X-ray CT apparatus in accordance with either one of eighth to eleventh aspects, in which the image quality adjustor means changes the image quality to a predetermined quality by using an image filter to be convoluted onto the tomographic image.

In the X-ray CT apparatus in accordance with the above twelfth aspect, the optimum image quality for each organ can be achieved by specifying an image quality by adjusting the image filter.

In thirteenth aspect, the present invention provides an X-ray CT apparatus in accordance with either one of eighth to twelfth aspects, in which the image quality adjustor means changes the image quality to a predetermined quality by using a reconstruction function to be convoluted onto the projection data.

In the X-ray CT apparatus in accordance with the above thirteenth aspect, the optimum image quality for each organ can be achieved by specifying an image quality by adjusting the reconstruction function.

In fourteenth aspect, the present invention provides an X-ray CT apparatus in accordance with thirteenth aspect, in which image quality selector means convolutes a plurality of reconstruction functions each different from other to the preprocessed projection data; back projects each of projection data having a reconstruction function convoluted to form a plurality of tomographic images; performs, to each specific area of tomographic image, a weighted addition of a plurality of tomographic images created by said back projection means to form one tomographic image.

In the X-ray CT apparatus in accordance with the above fourteenth aspect, the most optimized image quality can be achieved for every organ by adjusting the image quality by means of weighted addition of a plurality of tomographic images.

The image quality can be optimized for each area or each organ on the tomographic image by the reconstruction function and image filter.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic block diagram illustrating an X-ray CT apparatus in accordance with one preferred embodiment of the present invention;

FIG. 2 is a schematic diagram illustrating the processing flow in accordance with one preferred embodiment of the present invention;

FIG. 3 is a schematic diagram illustrating the settings for each area in accordance with the present invention;

FIG. 4 is a schematic diagram illustrating the switching of weighting coefficient for each area in accordance with the present invention; and

FIG. 5 is a schematic diagram illustrating the display screen for entering the weighting coefficients and thresholds of the reconstruction functions and image filters for each area in accordance with the present invention.

### DETAILED DESCRIPTION OF THE INVENTION

The present invention will be described in greater details herein below with reference to the embodiment shown in the drawings. It should be noted that the embodiment described herein below are presented merely for the illustration and are not intended to limit.

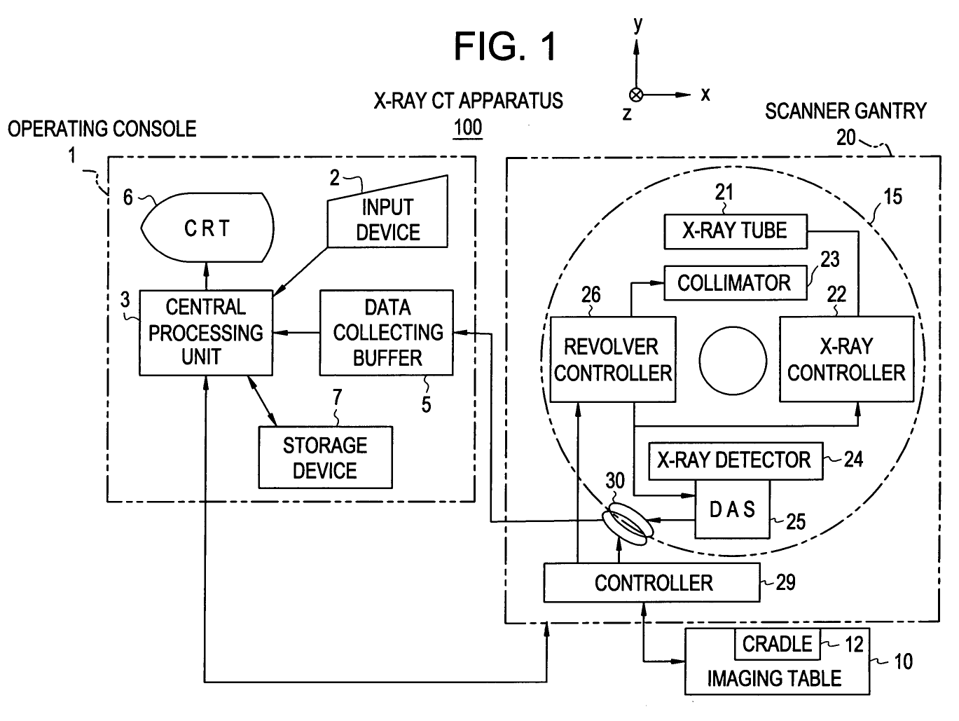

FIG. 1 shows a schematic block diagram of an X-ray CT apparatus in accordance with an embodiment of the present invention. The X-ray CT apparatus 100 includes an operating console 1, an imaging table 10, and a scanning gauntry 20.

The operating console 1 includes an input device 2 for receiving the input from the operator, a central processing unit 3 for performing such operations as image reconstruction processing, a data acquisition buffer 5 for acquiring projection data obtained from the scanning gauntry 20, a CRT 6 for displaying a CT image reconstructed from the projection data, and a storage device 7 for storing programs, data, and X-ray CT images.

The imaging table 10 includes a cradle 12 for carrying an object thereon into and out of the bore of the scanning gauntry 20. The cradle 12 is moved up and down and linearly translated by a motor built in the imaging table 10.

The scanning gauntry 20 includes an X-ray tube 21, an X-ray controller 22, a collimator 23, an X-ray detector 24, a data acquisition system DAS 25, a revolver controller 26 for revolving the X-ray tube 21 around the axis of the object, and a controller 29 for sending and receiving control signals to and from the operating console 1 and imaging table 10.

The structure of the X-ray CT system in accordance with this embodiment is as above. In this X-ray CT system, the acquisition of projection data may be performed as follows.

With an object positioned within the bore of the revolver 15 of the scanning gauntry 20, and with the position in the z-axis secured, X-ray beam from the X-ray tube 21 is irradiated to the object (X-ray projection), and transmitted X-ray is detected by the X-ray detector 24. Then, the detection of transmitted X-ray is performed while rotating the X-ray tube 21 and the X-ray detector 24 around the object (in other words varying the projection angle (view angle)) for a plurality of view directions N (for example N=1,000) to acquire data of 360 degrees.

The transmitted X-ray thus detected will be converted to digital value of projection data in the DAS 25, and the data will be transferred through the data collecting buffer 5 to the operating console 1. This operation completes one “scan”. Thereafter, the scanning position is translated sequentially along with the z-axis by a predetermined amount, another scan is performed. This type of scanning is referred to as “conventional scan” (or “axial scan”). Another type of scanning, so-called “helical scan” can be performed by translating the imaging table 10 at a predetermined speed in synchronism with the move of projection angle to acquire projection data (the X-ray tube 21 and the X-ray detector 24 revolves around the object as a helicoid). The present invention can be applied to both conventional and helical types of scanning.

The operating console 1 is used to store the projection data transferred from the scanning gauntry 20 to the fixed disk HDD in the central processing unit 3 as well as to perform a convolution operation with a predetermined reconstruction function and back projection processing to reconstruct tomographic images. The operating console 1 is capable of reconstructing a tomographic image on real-time basis from the projection data sequentially transferred from the scanning gauntry 20 during scanning to always display the current tomographic image on the CRT 6. It is also capable of performing image reconstruction by retrieving the projection data stored in the fixed disk HDD.

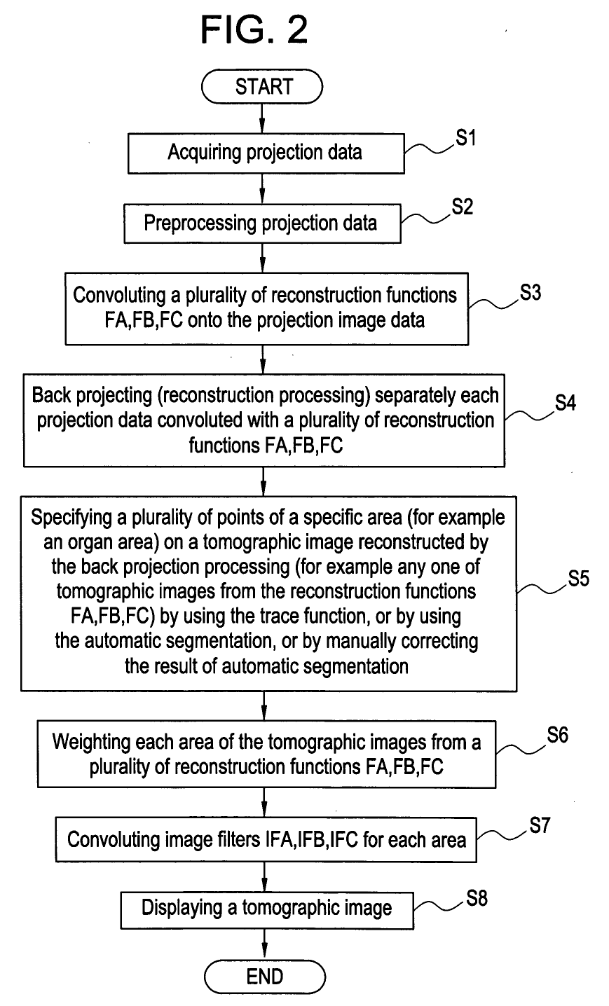

The processing operation by the operating console 1 in accordance with the present embodiment will be described with reference to illustration shown in FIG. 2.

FIG. 2 shows the illustration of the processing flow in the operating console 1 in accordance with the present embodiment. The program corresponding to the flow chart is included in an image processing program installed on the storage device 7 comprised of a hard disk drive HDD and the like, and is launched by the central processing unit 3.

In step S1, the data acquisition system, X-ray tube 21, X-ray detector 24, and DAS 25 of the scanning gauntry 20 are used to acquire projection data through the slip ring 30.

In step S2, a predetermined preprocessing on thus acquired data is performed, including logarithmic transformation, beam hardening correction, sensitivity correction of the X-ray detectors and so on.

In step S3, a plurality of reconstruction functions for example three reconstruction functions FA, FB, and FC each different from other, are convoluted onto the preprocessed projection data. In the present embodiment, for the reconstruction functions FA, FB, and FC, a reconstruction function FA for smoothing the image, a reconstruction function FB for sharpening the image, and a reconstruction function FC for performing the in-between of those are used. In such a manner, three projection data each convoluted with one of three reconstruction functions FA, FB, and FC are obtained.

In step S4, each projection data convoluted with one of three reconstruction functions FA, FB, and FC is back projected. This yields three tomographic images.

In step S5, either one tomographic image from within tomographic images reconstructed by the back projection, more specifically from those reconstruction functions FA, FB, and FC, is used as reference to apply the trace function to a specific area (for example an organ area) on the tomographic image, by specifying a plurality of points, or by using the automatic segmentation. The area can also be specified by manually correcting the result from the automatic segmentation.

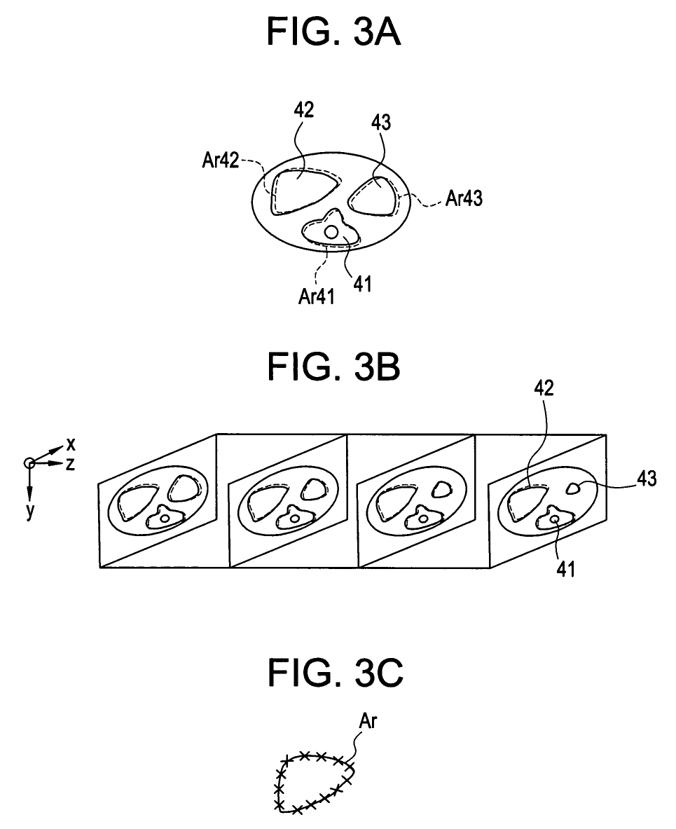

FIG. 3(a) shows a tomographic image at one slice position, FIG. 3(b) shows a tomographic image obtained at a plurality of continuous slice positions, and FIG. 3(c) shows a schematic illustration describing the trace function.

In FIGS. 3(a) and 3(b), an exemplary tomographic image is shown with bone 41, organs 42 and 43. In step S5, the automatic segmentation or the user using the trace function specifies an area Ar41 corresponding to the bone 41, areas Ar42 and Ar43 corresponding to the organs 42 and 43. Alternatively, the user corrects manually the result from the automatic segmentation to specify areas Ar41, Ar42, and Ar43.

The automatic segmentation specifies areas by automatically recognizing organs and bones, based on the CT values in the tomographic image, by an image-processing program stored in the storage device 7. The trace function, as shown in FIG. 3(c), for example, is used by the user observing the tomographic image, for clicking some points on the interface between organs and bones (shown as crosses in the drawing), to connect these points with a straight line or a curved line (for example a spline interpolation) to create a closed area Ar.

Each area surrounding a bone or an organ in the tomographic image is thus specified as above.

In step S6, one tomographic image is synthesized by weighting the tomographic images of a plurality of reconstruction functions FA, FB, and FC for each of areas Ar41 to Ar43. More specifically, three reconstructed (tomographic) images, each reconstructed using one of three reconstruction functions, can be obtained from the processing of step S4. Based on the CT values of areas Ar41 to Ar43 for example, the most suitable reconstructed images for each of areas Ar41 to Ar43 are combined.

FIG. 4 shows a graph illustrating the deviation of weighting coefficient for each reconstruction function, used for the weighted addition of the tomographic image of a plurality of reconstruction functions FA, FB, and FC for each area in step S6.

For the CT value thresholds Th1, Th2, Th3, Th4, Th5, Th6, the tomographic image of respective reconstruction functions are switched. The sum of all weighted coefficients is 1 in the range of all CT values. For example, the reconstructed image from the reconstruction function FA is used for the area of higher CT values (more than threshold Th6), the reconstructed image from the reconstruction function FB is used for the area of lower CT values (more than threshold Th2 and less than Th3), and the reconstructed image from the reconstruction function FC is used for the intermediate area of CT values (more than threshold Th4 and less than Th5). The boundary areas, in other words the areas between Th1 and Th2, Th3 and Th4, and Th5 and Th6 are synthesized by linear weighting of two reconstructed images as shown in FIG. 4.

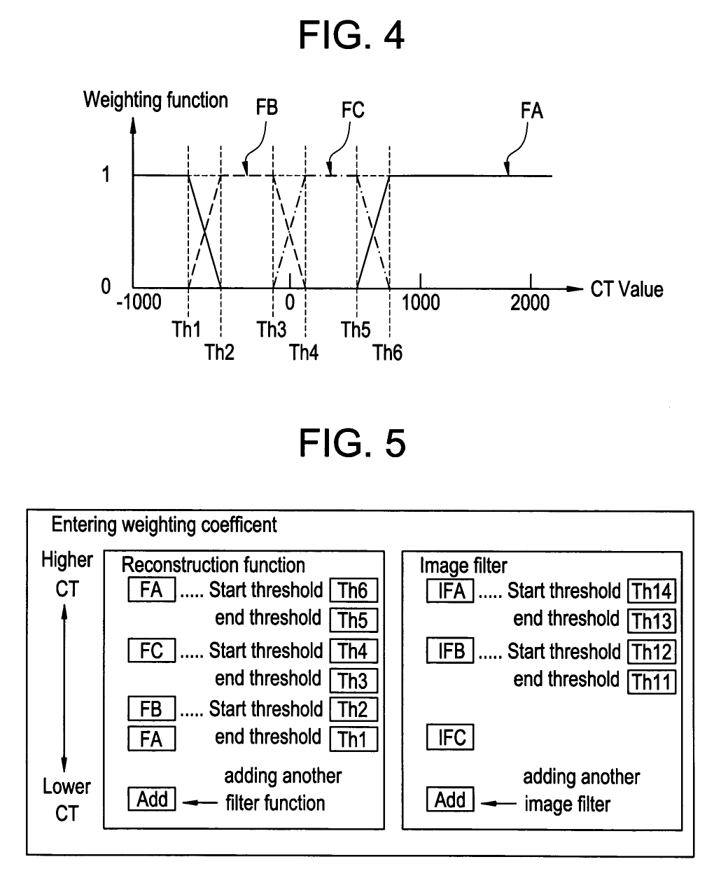

FIG. 5 shows a schematic diagram illustrating a screen for entering parameters used for weighting the reconstructed images using a plurality of reconstruction functions FA, FB, and FC for each area in step S6.

In FIG. 5, there are shown a screen for entering thresholds Th1, Th2, Th3, Th4, Th5 and Th6 of the weighting coefficient for each of reconstruction functions, and another screen for pre-entering the thresholds Th11, Th12, Th13, and Th14 of the weighting coefficient for each of image filters used in step S7 for convoluting a plurality of image filters IFA, IFB, and IFC for each of areas Ar41 to Ar43.

In step S7, image filters IFA, IFB, and IFC are convoluted for each of areas. More specifically, the type of image filter to be convoluted is changed for each of areas on the synthesized tomographic image. For example, the image filter IFA is convoluted on the area from the reconstructed image using the reconstruction function FA, the image filter IFB is convoluted on the area from the reconstructed image using the reconstruction function FB, and the image filter IFC is convoluted on the area from the reconstructed image using the reconstruction function FC.

In step S8, a tomographic image is displayed. One tomographic image with the optimized image quality for each of specific areas such as organs is thus displayed.

As have been described above, in accordance with the present embodiment, image quality for each area or each organ in the tomographic image can be optimized by creating a plurality of reconstruction images reconstructed by using reconstruction functions each different from other, and by specifying specific areas such as organs and the like included in the tomographic images by means of the trace function or the automatic segmentation, and by synthesizing an image from reconstruction images for each specified area.

Although in the present embodiment, an example using three reconstruction functions FA, FB, and FC and three image filters IFA, IFB, and IFC has been described, more or less number of functions and filters can be equally used. Alternatively, the image quality can be optimized by solely using the reconstruction function. Alternatively, the image quality can be optimized with one reconstruction function followed by a plurality of image filters.

In the above embodiment the controlling processing in the operating console 1 achieves the functional means of the present invention, including the preprocessing means, reconstruction function convolution means, back projection means, area specifying means, and image quality adjustment means. The scanning gauntry 20 corresponds to the X-ray data acquisition means.

In addition, the program being installed to the computer for providing thereby the functional processing in accordance with the present invention, as well as the recording medium for storing the program are also to implement the present invention. More specifically, the claims of the present invention should include the program for achieving the functional processing in accordance with the present invention, as well as the computer readable recording medium for storing the program.

The recording medium for storing the program may include for example, flexible disks, optical disks (such as CD-ROM, CD-R, CD-RW, DVD and the like), magneto-optical disks, magnetic tapes, memory cards, and the like.

As a means of supplying the program, a mode is included in which the program in accordance with the present invention can be obtained by file transfer on the Internet.

Many widely different embodiments of the invention may be configured without departing from the spirit and the scope of the present invention. It should be understood that the present invention is not limited to the specific embodiments described in the specification, except as defined in the appended claims.

1. An X-ray CT image processing method, comprising the steps of:
irradiating X-ray to an object for acquiring projection data of the X-ray passing through the object;
preprocessing the projection data thus acquired;
convoluting a reconstruction function onto thus preprocessed projection data;
reverse projecting the projection data having the reconstruction function convoluted to reconstruct the image of tomography;
specifying a specific area within the tomographic image; and
changing the image quality of said specific area to a predetermined quality.

2. An X-ray CT image processing method according to claim 1, in which said step of specifying the specific area within the tomographic image further comprising the substep of:
specifying an area of specific organ.

3. An X-ray CT image processing method according to claim 1, in which said step of specifying the specific area within the tomographic image further comprising the substep of:
performing the area selection by using a trace function for determining the specified area by specifying a plurality of points on the tomographic image.

4. An X-ray CT image processing method according to claim 1, in which said step of specifying the specific area within the tomographic image further comprising the substep of:
performing the area selection either by means of automatic segmentation, or by manually correcting the result of the automatic segmentation.

5. An X-ray CT image processing method according to claim 1, in which said step of changing the image quality of said specific area to a predetermined quality comprising the substep of:
changing the image quality to a predetermined quality by using an image filter to be convoluted onto the tomographic image.

6. An X-ray CT image processing method according to claim 1, in which said step of changing the image quality of said specific area to a predetermined quality comprising the substep of:
changing the image quality to a predetermined quality by using a reconstruction function to be convoluted onto the projection data.

7. An X-ray CT image processing method according to claim 6, in which said step of changing the image quality of said specific area to a predetermined image quality comprising the substeps of:
convoluting a plurality of reconstruction functions each different from other to the preprocessed projection data;
back projecting each of projection data having a reconstruction function convoluted to form a plurality of tomographic images; and
to each specific area of tomographic image, performing a weighted addition of a plurality of tomographic images created by said back projection means to form one tomographic image.

8. An X-ray CT apparatus, comprising:
an X-ray data acquisition device for irradiating X-ray to an object to acquire the projection data of X-ray passing through the object;
a preprocessing device for preprocessing the projection data acquired by said X-ray data acquisition device;
a reconstruction function convoluting device for convoluting a reconstruction function onto the preprocessed projection data;
a back projection device for back projecting the projection data having the reconstruction function convoluted so as to reconstruct a tomographic image;
an area selector device for selecting a specific area of tomographic image; and
an image quality adjustor device for adjusting the image quality of said specific area to a predetermined quality.

9. An X-ray CT apparatus according to claim 8, in which
said area selector device specifies an area of a specific organ.

10. An X-ray CT apparatus according to claim 8, in which
said area selector device performs the area selection by using a trace function for determining the specified area by specifying a plurality of points on the tomographic image.

11. An X-ray CT apparatus according to claim 8, in which
said area selector device performs the area selection either by device of automatic segmentation, or by manually correcting the result of automatic segmentation.

12. An X-ray CT apparatus according to claim 8, in which
said image quality adjustor device changes the image quality to a predetermined quality by using an image filter to be convoluted onto the tomographic image.

13. An X-ray CT apparatus according to claim 8, in which
said image quality adjustor device changes the image quality to a predetermined quality by using a reconstruction function to be convoluted onto the projection data.

14. An X-ray CT apparatus according to claim 13, in which
said image quality selector device convolutes a plurality of reconstruction functions each different from other to the preprocessed projection data;
back projects each of projection data having a reconstruction function convoluted to form a plurality of tomographic images;
performs, to each specific area of tomographic image, a weighted addition of a plurality of tomographic images created by said back projection device to form one tomographic image.
